(19) United States
(12) Patent Application Publication
(10) Pub. No.: US 2005/0203529 A1
(43) Pub. Date: Sep. 15, 2005

(54) Minimally-invasive method for performing spinal fusion and bone graft capsule for facilitating the same

(76) Inventors: Frank H. Boehm JR., Utica, NY (US); Benedetta Delorenzo Melnick, Rome, NY (US)

Correspondence Address: DILWORTH & BARRESE, LLP, 333 EARLE OVINGTON BLVD., UNIONDALE, NY, 11553, US

(21) Appl. No.: 11/071,727

(22) Filed: Mar. 3, 2005

Related U.S. Application Data
(60) Provisional application No. 60/549,695, filed on Mar. 3, 2004.

(51) Int. Cl. A61B 17/56

(52) U.S. Cl. 606/86

(57) ABSTRACT

A method for performing a spinal fusion procedure between the transverse processes of two adjacent vertebra is disclosed. The method includes creating a vascularized bone flap at each of the transverse processes and introducing a bone graft delivery device to the site between the transverse processes and the vascularized bone grafts. A novel bone graft delivery system and device is also disclosed.

### CROSS-REFERENCE TO RELATED APPLICATION

This application is based on, and claims priority to, U.S. Provisional Patent Application No. 60/549,695 filed on Mar. 3, 2004, the entire contents of which are hereby incorporated by reference.

### BACKGROUND

1. Field of the Invention

The present invention relates to a minimally invasive method for performing spinal fusion procedures. In particular, the invention relates to a method for subcutaneously supplementing a stabilization procedure using pedicle screws and rods to stabilize adjacent vertebra, by utilizing a minimally-invasive-posterior-approach method of implanting bone growth material in the form of bone graft capsule to create a bone bridge between the adjacent vertebra.

2. Description of the Related Art

Surgical treatment for spine disease may involve stabilization of the spine following disc removal and implantation of artificial discs. A common stabilization technique provides for screws to be secured into the pedicles of adjacent vertebrae, with the screws then being joined to each other by a connecting cross-piece such as a rod or plate to stabilize adjacent vertebrae. The procedure is typically done on both sides of the spinal column, to stabilize both sides of the adjacent vertebrae, and may be done over a series of vertebra, to stabilize multiple levels. There are a number of ways to secure the screws to the rod. In general, they include some form of a nut or bolt secured to the screw head to lock the rod or plate in place.

After the disc has been removed, an artificial disc may be implanted, a bone matrix may be injected, or both, to facilitate fusion between the adjacent vertebrae. Since the injectable material is somewhat viscous, it typically works only when injected into a contained area or site, thus limiting its applicability to fusions in the intervertebral disc space. In addition, while the pedicle screw and rod system is sufficient to stabilize the vertebra while the vertebra fuse, over time the screw and rod system may fail, which places a burden on the fusion site, particularly if the fusion was not successful or only partially successful. Even if successful, the fusion usually secures a minimal surface area of the bone surface between the adjacent vertebrae.

Spinal fusion is a surgical technique that ultimately leads to the formation of a bony bridge, or union, between two or more vertebrae of the spine. Many efforts are now being dedicated towards developing techniques for accomplishing spinal fusion utilizing minimally invasive techniques. This conundrum refers to a rapidly expanding field of surgery in which the objective of the procedure can be accomplished using much smaller incisions, more precisely-placed instruments, and minimizing the involvement of tissues and structures not directly related to the pathology and surgical procedure. The benefits of such an approach are obvious and self-explanatory, and include reduction of operating room time, reduction of anesthesia, limiting the opportunity for bleeding, infection, and other well-known complications of surgery. In addition, these techniques offer substantial cost savings to a system, which is already over-, burdened from a fiscal perspective.

Many spinal pathologies are already being approached using such techniques. The concept of “minimally-invasive percutaneous discectomy,” has been used in one form or another for over two decades. These techniques take full advantage of the advances in radiologic techniques, such as the use of real-time fluoroscopic guidance using either a C-arm or CT scanning. Such techniques rely on “indirect,” visualization, and anticipate, to a certain extent, a standard anatomic arrangement.

Another field of minimally-invasive techniques that have been introduces utilizes endoscopic techniques, or the use of limited incisions in combination with the use of small, fiberoptically enhanced visualizing devices with permit the surgeon to directly visualize the surgical field, although it may be substantially modified from the anatomy which is classically seen in so-called “open” procedures. Numerous procedures utilizing such techniques have now been introduces, including removal of herniated disc fragments, as well as techniques for posterolateral fusion, techniques for transforaminal, interbody fusion techniques (T-LIF), and techniques for anterior interbody fusion utilizing (ALIF) placement of interbody fusion cages.

Because of the novel but at times disorienting visualization associated with endoscopic techniques, many surgeons have become more comfortable with radiographically-guided techniques. More to the point, it is felt by many that, in the end, that there is a role for both of these techniques in the armamentarium of the spinal surgeon.

Therefore, a need exists for a method to fuse adjacent vertebrae at a more stable and secure location, such as at the transverse processes, to provide greater stability and strength to the fusion. A need also exists for a bone graft device to facilitate such a fusion, which is easy to install and which enhances the possibility of successful fusion.

### SUMMARY

The present invention has been made in view of the above problems associated with the prior art techniques and methods, and provides a minimally invasive surgical method for effecting spinal fusion. The method of the present invention may be performed to supplement a spinal stabilization procedure, or may be performed to supplement a disc replacement procedure and fusion. The method of the present invention may also be done as a stand-alone procedure, to fuse adjacent vertebrae not requiring a discectomy of stabilization procedure.

The present invention also provides a novel bone graft delivery mechanism to facilitate a bony fusion between adjacent vertebrae at a location that will provide more stability and strength to the spine at the fusion site. The delivery mechanism consists of a string of capsules that contain at least one of cadaveric bone, autologous bone, allographic bone obtained from cadaveric specimens, demineralized bone material, bone morphogenic protein (BMP), hormonal like substances which promote bone growth/fusion and hydroxyapatite, preferably encapsulated in a biocompatible and biodegradable gelatinous envelope, which rapidly dissolves at the fusion site. Once the encasing material dissolves, the contents of the capsules come into contact with one another as well as with the biologic components of the vertebrae to be fused.

The preferred fusion site is between two transverse processes of adjacent vertebrae, and may also be done between the last lumbar transverse process and the ala of the sacrum.

It is also contemplated that an autograft may be harvested from the patient prior to the surgery, morselized, and inserted into empty shells of the spheres or capsules, so that the patient's own bone material may be utilized.

The spheres we connected or joined into an assembly or “string,” resembling a string of beads. The connections in this string are also composed of absorbable material, again, not being absorbed until exposed to the internal milieu of the body. This assembly facilitates positioning the bone graft material into a plane or space in the soft tissues between the transverse processes of the contiguous vertebrae to be fused. In the special case of a fusion between the last lumbar vertebra and the sacrum, the plane referenced above would be created between the transverse process and the sacral ala. In either case, these tissue planes are analogous to the “lateral gutters” which are classically created by a surgeon in an open procedure. Filling these planes with bone graft material will then result in a lateral-mass fusion.

The method of the present invention divides the transverse processes and ala in a coronal plane. A method for creating a plane in the soft tissues between these osseous structures is also provided. The bone graft delivery system can be juxtapositioned into the aforementioned soft-tissue plane. This is accomplished by utilizing the same gelatinous material used to encase the bone material in a gelatinous matrix that would also connect these spheres. This permits passage of a large amount of bone material into the space in the tissue planes. A leading end of this assembly or “string” has an area that can be grasped by a device designed to be passed into the tissue plane from an incision separate from the incision through which the assembly is passed. The leading end of the assembly is grasped, and in this way guided into the proposed site fusion.

The bone graft capsules of the present invention facilitate placement of the graft onto the plane between the adjacent transverse processes or between the transverse process and sacral ala being fused. A cutting tool to perform an osteotomy of the transverse processes and/or sacral ala is introduced under fluoroscopy from a position superior and inferior to the areas of fusion.

### BRIEF DESCRIPTION OF THE DRAWINGS

The above and other objects of the present invention will become more readily apparent from the following detailed description of preferred embodiments of the present invention, taken in conjunction with the accompanying drawings, in which:

FIG. 1 shows a posterior view of a pair of adjacent lumbar vertebra in which a guide needle is secured in a transverse process of one of the vertebra;

FIG. 2 shows a working channel set on a pedicle of each of the vertebra, for performing a stabilization procedure utilizing pedicle screws and rods, in accordance with an embodiment of the present invention;

FIG. 3 shows a portion of an inferior vertebrae of the pair of vertebrae, with the superior vertebrae deleted from the drawing for purposes of clarity, with a working channel in place in accordance with an embodiment of the present invention;

FIG. 4 shows a guide needle being advanced to the working channel in accordance with an embodiment of the present invention;

FIG. 5 shows a portion of the inferior vertebrae of FIG. 4 with the guide needle advancing towards the working channel in accordance with an embodiment of the present invention;

FIG. 6 shows the guide needle at the capture zone of the working channel;

FIG. 7 shows a portion of the inferior vertebrae of FIG. 6 with the needle at the capture zone;

FIG. 8 shows a slidable panel on the working channel being move to drive the tip of the needle into the transverse process;

FIG. 9 shows the slidable panel returned to its original position, with the needle being in the transverse process;

FIG. 10 shows a cannulated hand drill being advanced along the guide needle to the transverse process;

FIG. 11 shows the posterior view of the pair of vertebra with the drill being advanced along the guide needle;

FIG. 12 shows the drill enlarging the hole made by the guide needle;

FIG. 13 shows the posterior view of the pair of vertebra with the drill enlarging the hole made by the guide needle;

FIG. 14 shows a cannulated hand saw being advanced over the guide needle to the hole made by the drill in the transverse process;

FIG. 15 shows the posterior view of the pair of vertebra with the hand saw being advanced over the guide needle;

FIG. 16 shows the hand saw beginning the cutting process to perform the osteotomy on the transverse process, and shows the proposed course of the osteotomy;

FIG. 17 shows the hand saw cutting the transverse process;

FIG. 18 shows the posterior view of the pair of vertebra with the hand saw cutting the transverse process;

FIG. 19 shows the second portion of the osteotomy of the transverse process, in which the hand saw is rotated approximately 90°, and shows the proposed course of the vertical osteotomy;

FIG. 20 shows the posterior view of the pair of vertebra, with the proposed course of the osteotomy;

FIG. 21 shows the completed osteotomy;

FIG. 22 shows a grasping tool introduced over the guide needle to lift the flap of cut bone off the transverse process;

FIG. 23 shows the posterior view of the pair of vertebra in which the transverse process of the superior vertebrae has undergone the osteotomy, and the bone flap has been lifted off the transverse process;

FIG. 24 shows the posterior view of the pair of vertebra where the bone graft capsules of the present invention are being moved into place to begin the fusion process;

FIG. 25 shows the posterior view of the pair of vertebra where the bone graft capsules are in position over both transverse processes, and multiple capsule strings have been put in place to facilitate fusion;

FIG. 26 shows an embodiment of the bone graft capsules; and

FIG. 27 shows a cross-section of several of the individual capsules of FIG. 26.

### DETAILED DESCRIPTION

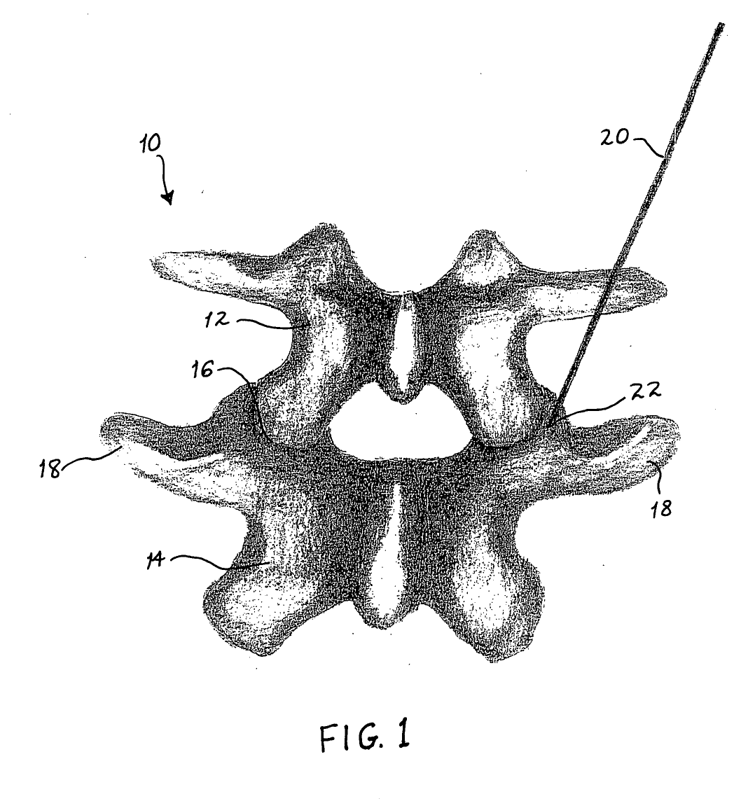

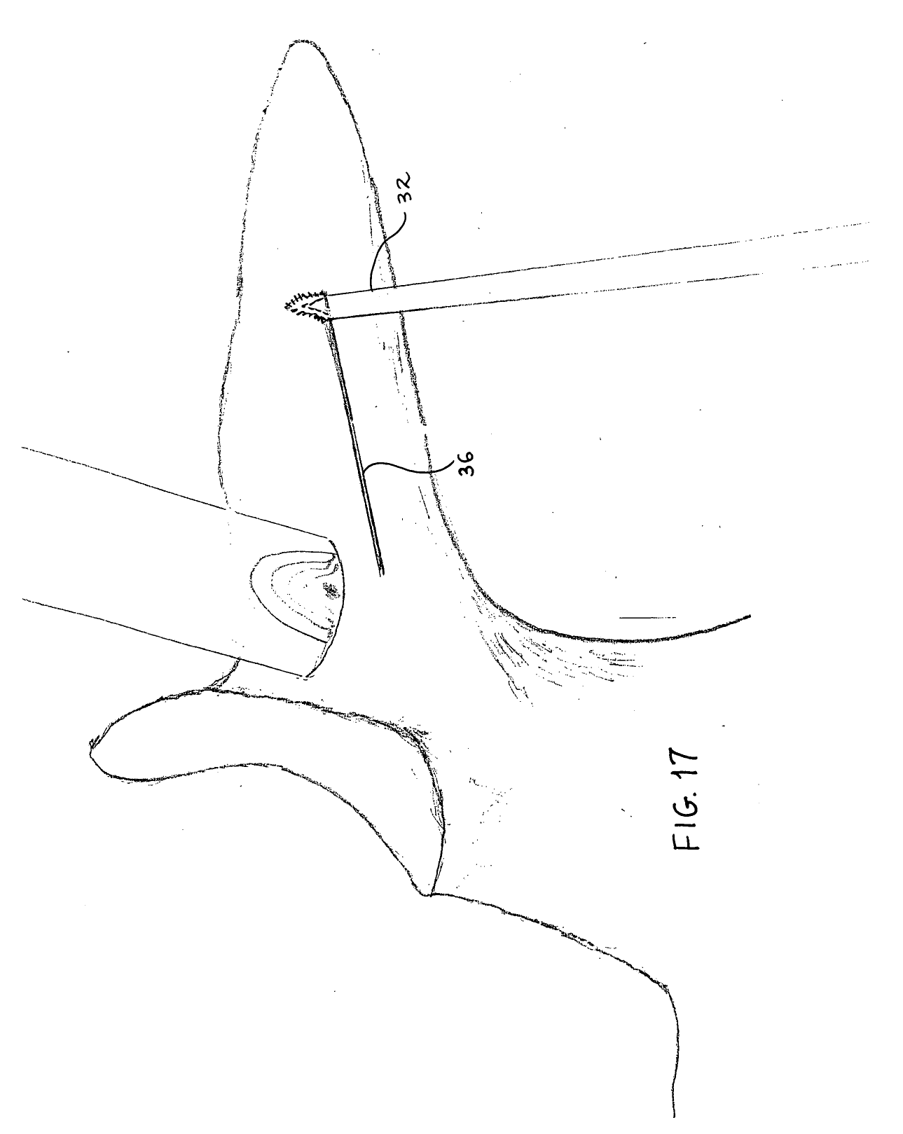

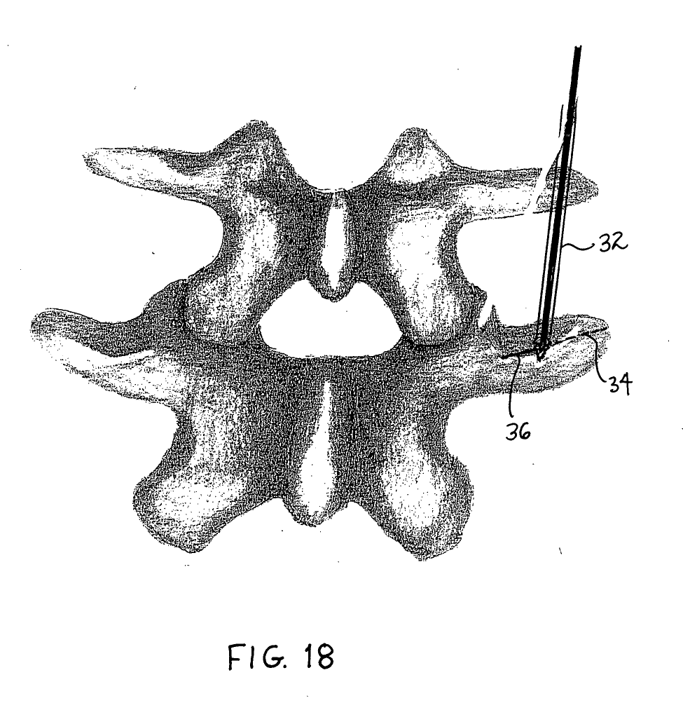

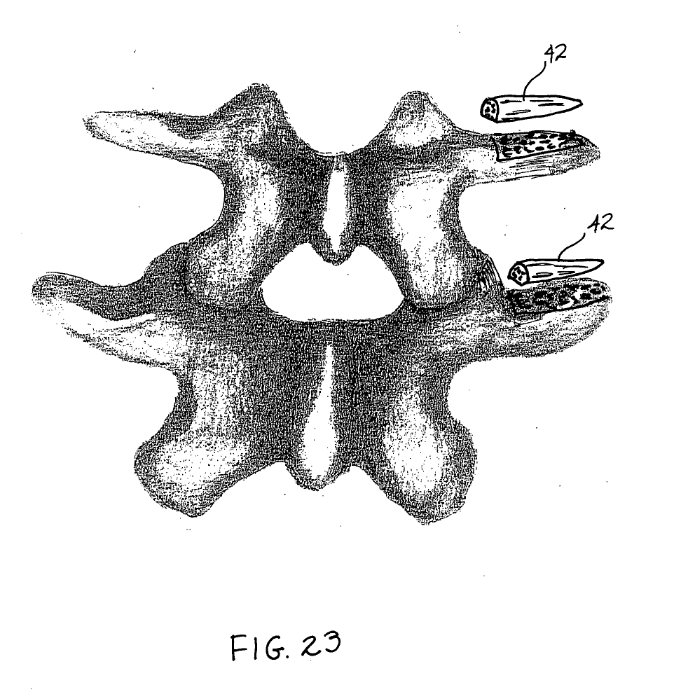

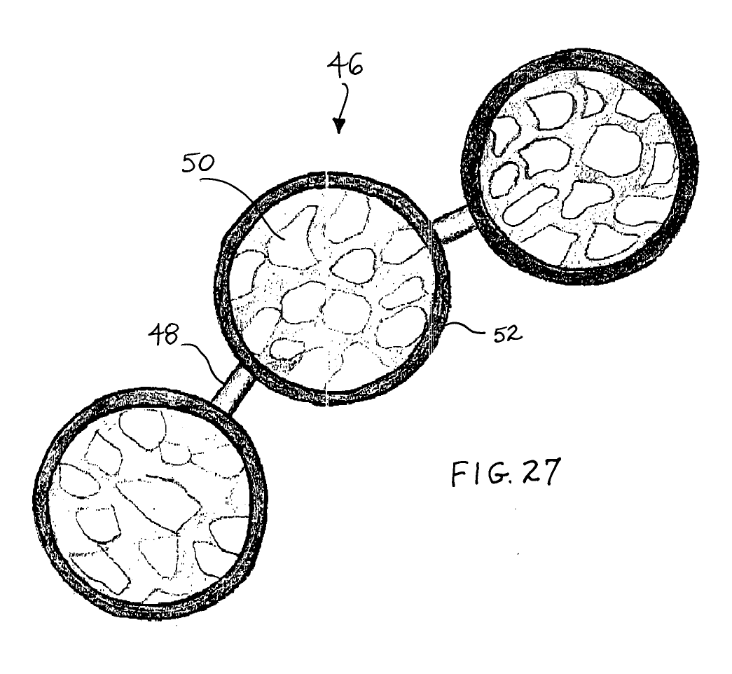

Referring now to the drawings, in which like reference numerals identify similar or identical elements throughout the many views, FIGS. 1-26 illustrate the minimally invasive fusion procedure of the present invention, while FIGS. 27 and 28 illustrate the novel bone graft capsules of the present invention, which facilitate the fusion procedure. The fusion procedure of the present invention can be carried out in many ways, but is preferably done through small incisions under fluoroscopy or other imaging procedure to view the surgical process. That is, the method of the present invention may be performed percutaneously, endoscopically, or even in a traditional “open” surgical procedure. Preferably, the method is performed percutaneously.

The fusion procedure preferably supplements a stabilization procedure, which, for example, provides pedicle screws and connecting rods between the pedicle screws. In such stabilization procedure, a series of dilators are passed over a guide needle through a small incision to the pedicle of the vertebrae. Each successive dilator has an inner diameter that is slightly larger than the outer diameter of the previous dilator, to enlarge the incision without cutting or tearing the tissue and muscles of the patient, thus minimizing trauma to the patient. Once a working channel of sufficient size is established, all the dilators are removed, with the exception of the outermost dilator, which becomes the working channel. A pedicle screw is then passed down the channel and secured to the vertebrae at the pedicle. The same is done for the adjacent vertebrae, and a connecting rod is the secured between the two screws. The procedure is the repeated on the other lateral side of the spinal column, to stabilize the pair of vertebra. Such a procedure is described in co-pending U.S. application Ser. No. 10/320,989, the entire contents of which are incorporated herein by reference.

While the present invention may be utilized to supplement the stabilization procedure, the fusion procedure may be performed on its own, that is, without the stabilization procedure, if necessary. As described below, the method of the present may utilize instrumentation that is used in the stabilization procedure, if the stabilization procedure is being concurrently performed, or the method may be performed without reliance on any instrumentation utilized in the stabilization procedure, for times when the fusion procedure is performed by itself on a patient requiring fusion only.

Turning now to the drawings, FIG. 1 shows a posterior view of a pair of vertebra 10, consisting of superior vertebrae 12 and inferior vertebrae 14. The vertebra are connected to each other at the facet joints 16, and extending from the vertebra are the transverse processes 18. While many fusion procedures fuse the bones of the vertebra 10 at the location of a disc that has been removed during a discectomy, the present invention provides for fusion between the transverse processes 18, which provides greater strength at the fusion site, and thus greater stability for the spinal column after fusion. In FIG. 1, a guide needle 20 has been passed through a small incision in the patient's back and is directed to the fusion site 22.

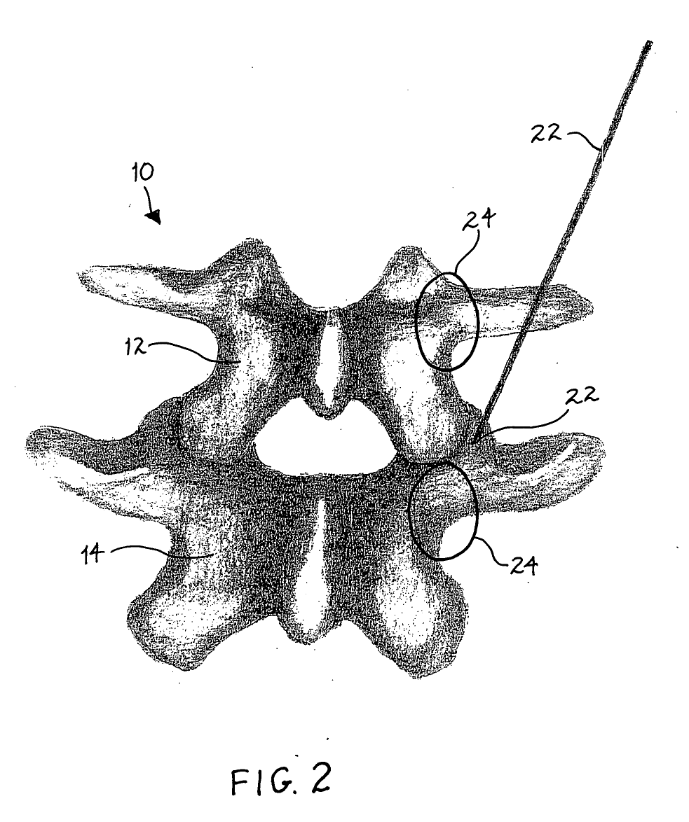

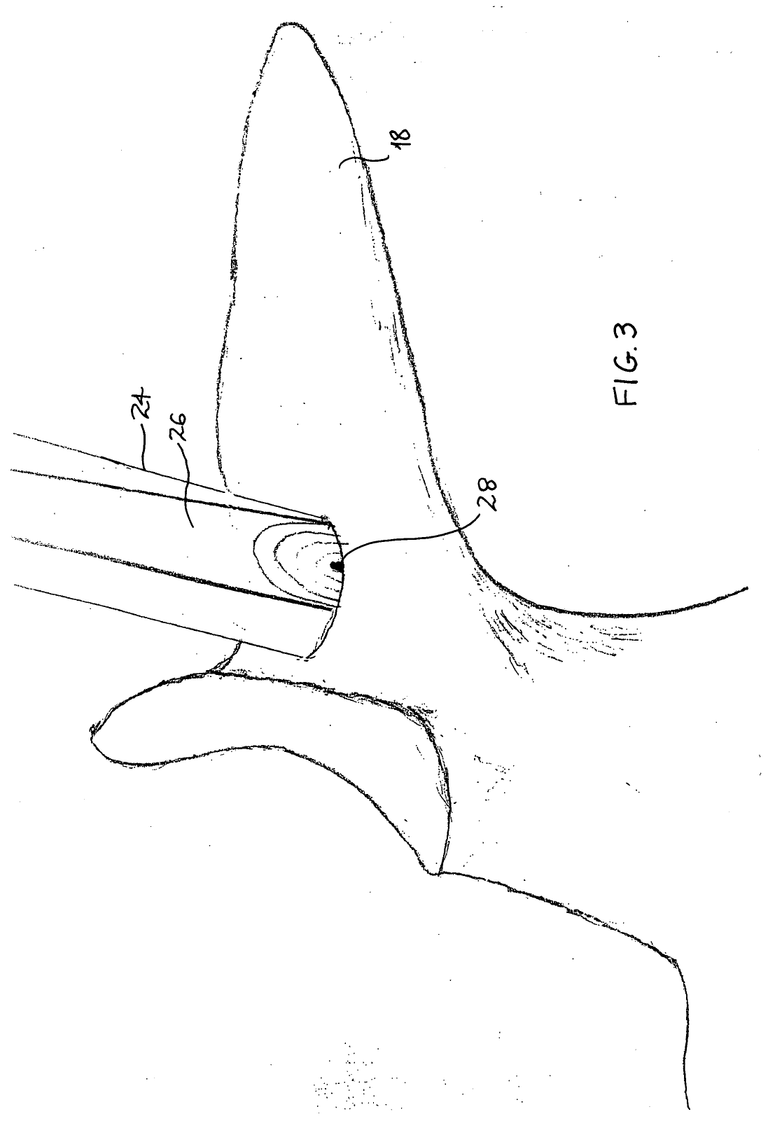

As has been described above, when the fusion procedure is performed to supplement a stabilization procedure, the method of the present invention may utilize instrumentation already in place from the stabilization procedure. As seen in FIGS. 2 and 3, a pair of working channels 24 remain in place. The novel dilator forming the working channel 24 is provided with slidable panel 26 which terminates in a notch or “capture zone” 28. Preferably, at least the area surrounding the capture zone 28 is radiopaque, for easy viewing under fluoroscopy, although the slidable panel 26 and/or the entire working channel 24 may be radiopaque also.

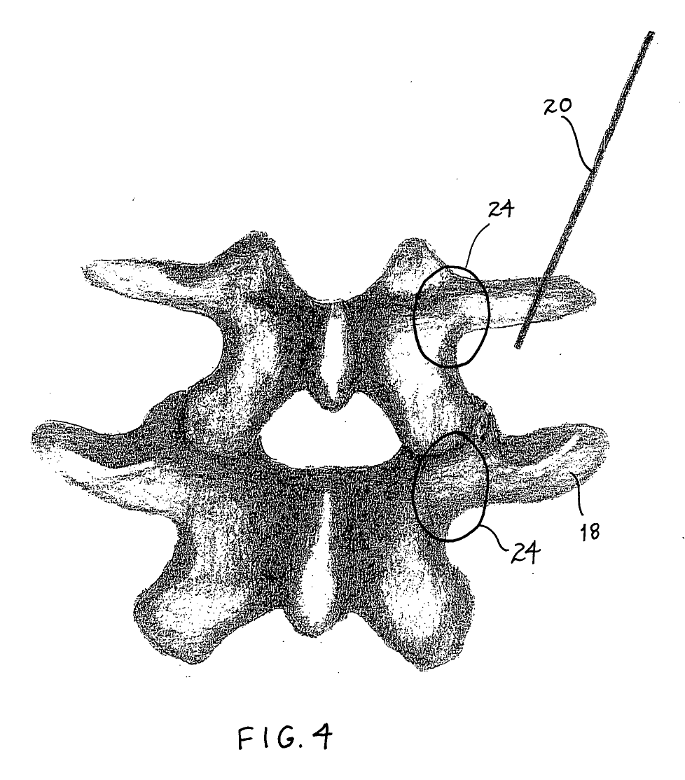

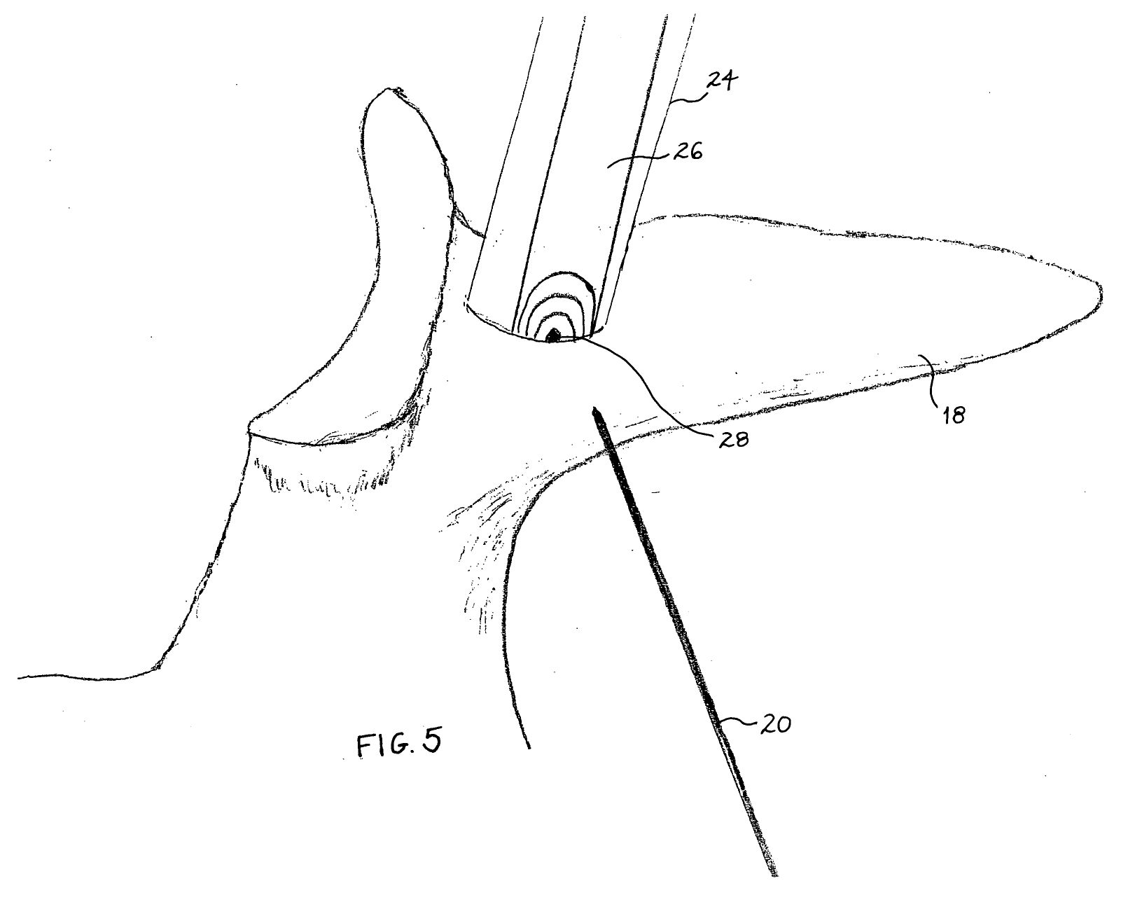

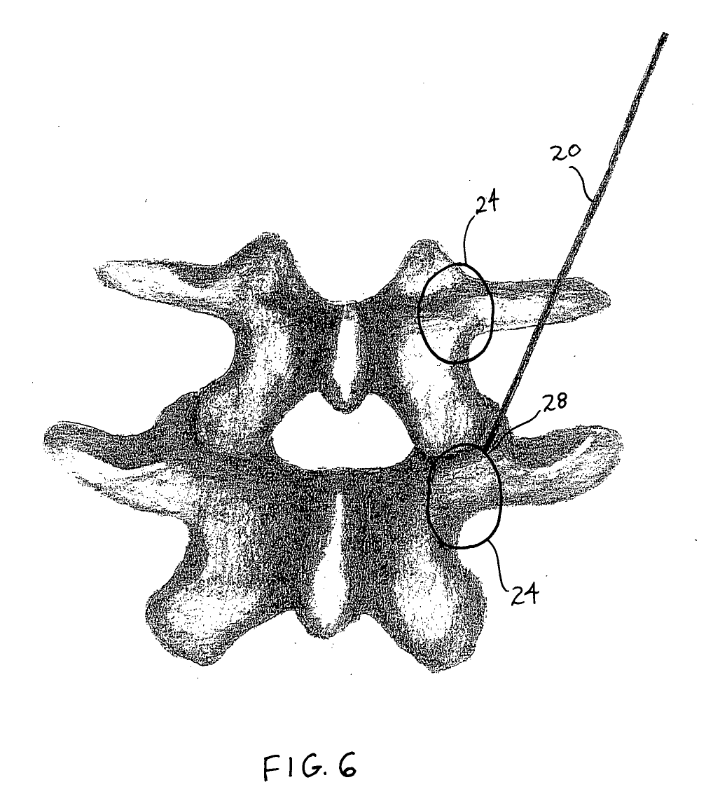

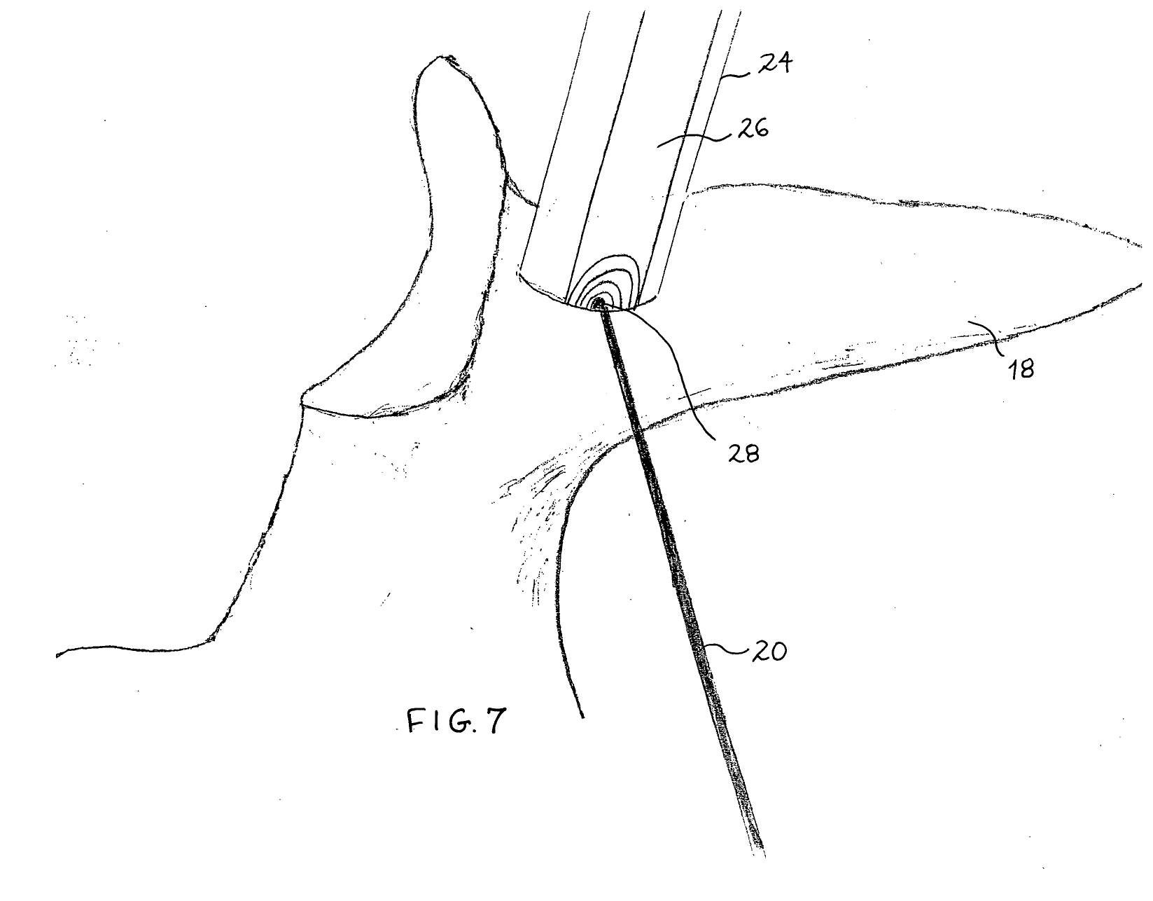

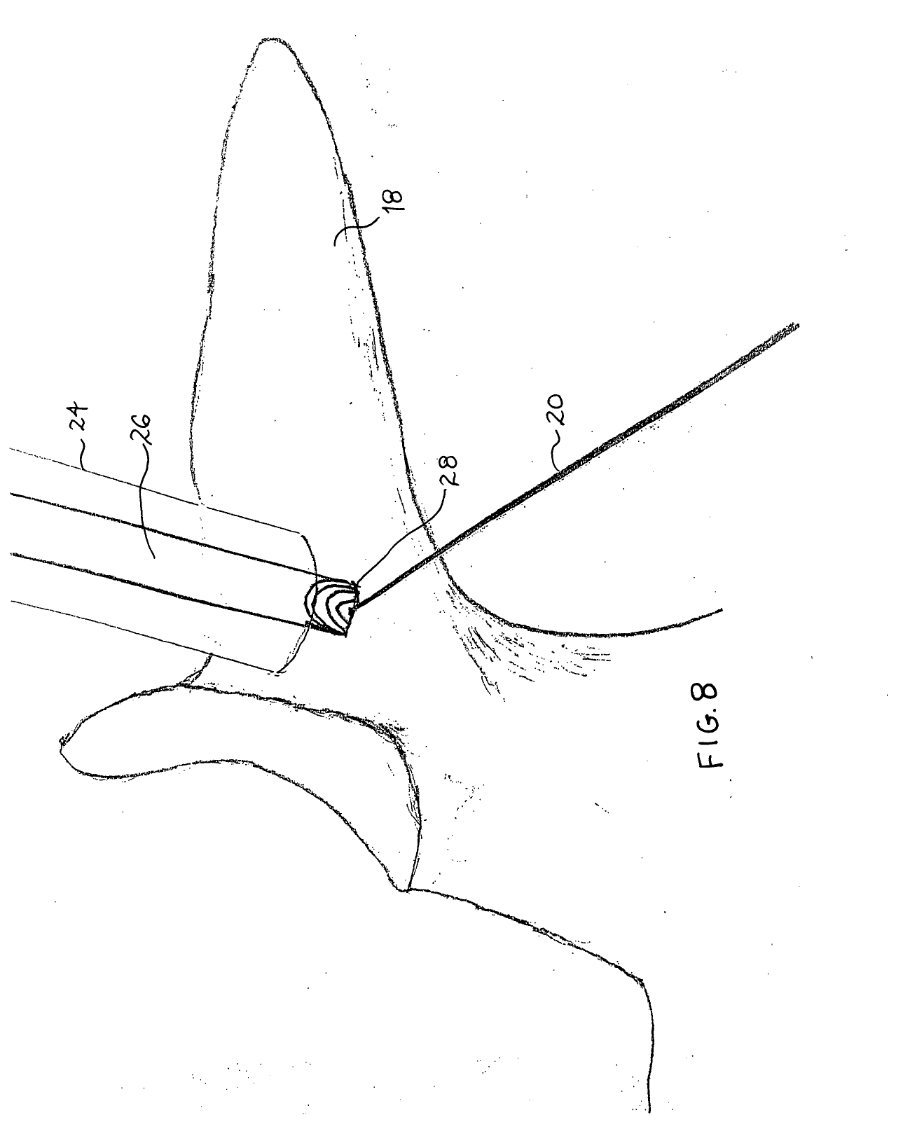

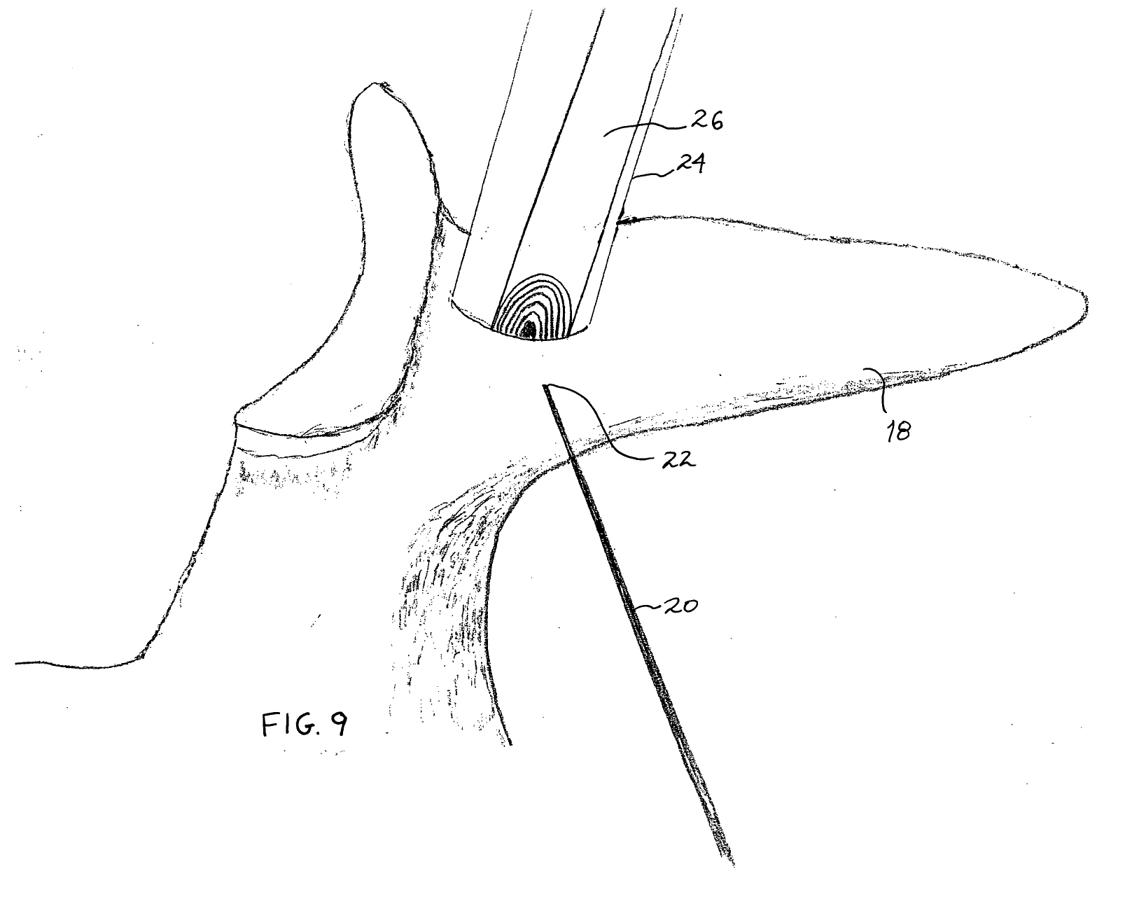

When utilizing the working channel 24, the surgeon advances the guide needle 20 to the fusion site 22 at the base of the working channel 24, as seen in FIGS. 4 and 5. When the guide needle reaches the capture zone 28, as seen in FIGS. 6 and 7, the surgeon manipulates the slidable panel 26 to advance the slidable panel 26 against the guide needle 20, to assist in driving the tip of the guide needle into the bone of the transverse process 18, as seen in FIG. 8. After the tip of the guide needle 20 is in place, the surgeon manipulates the slidable panel to return it to its original position, as seen in FIG. 9.

Of course, guide needle 20 may be forced into the bone of the transverse process 18 by hand pressure, when the working channel is not present, such as during a procedure involving fusion only.

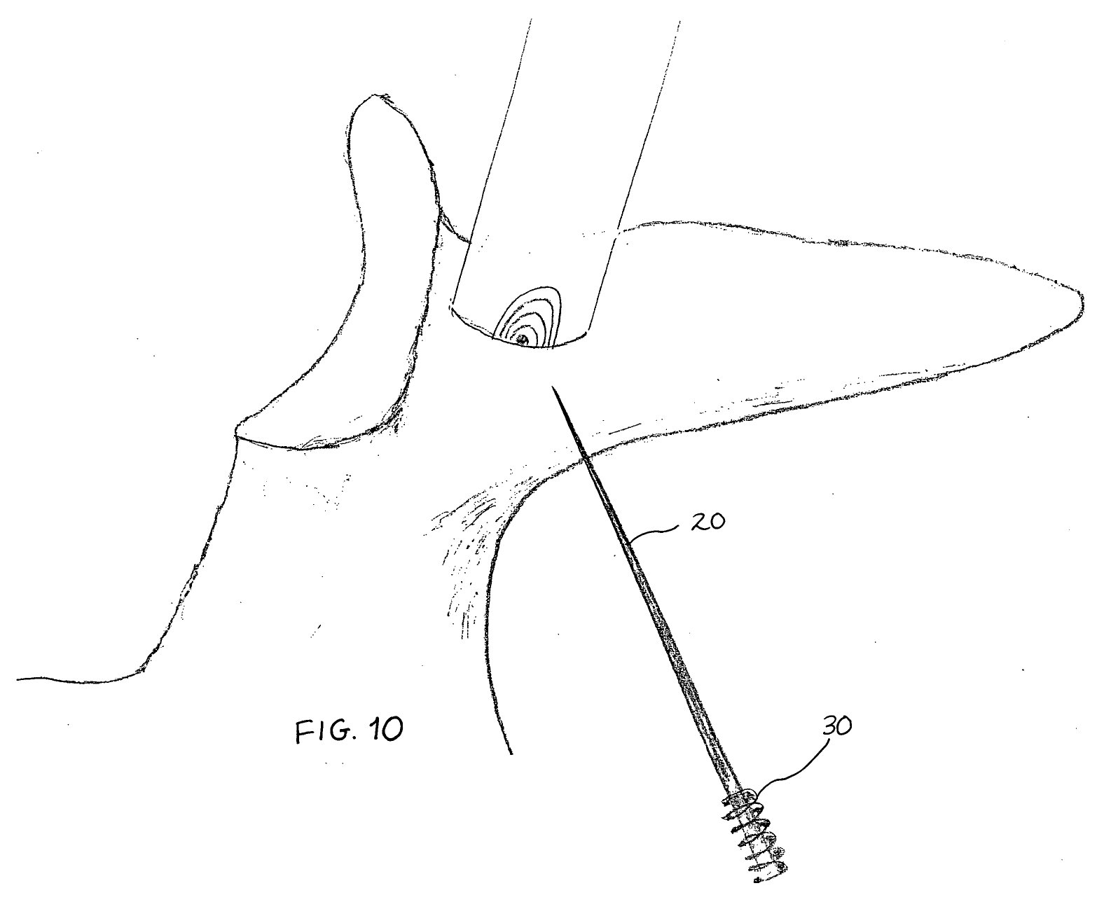

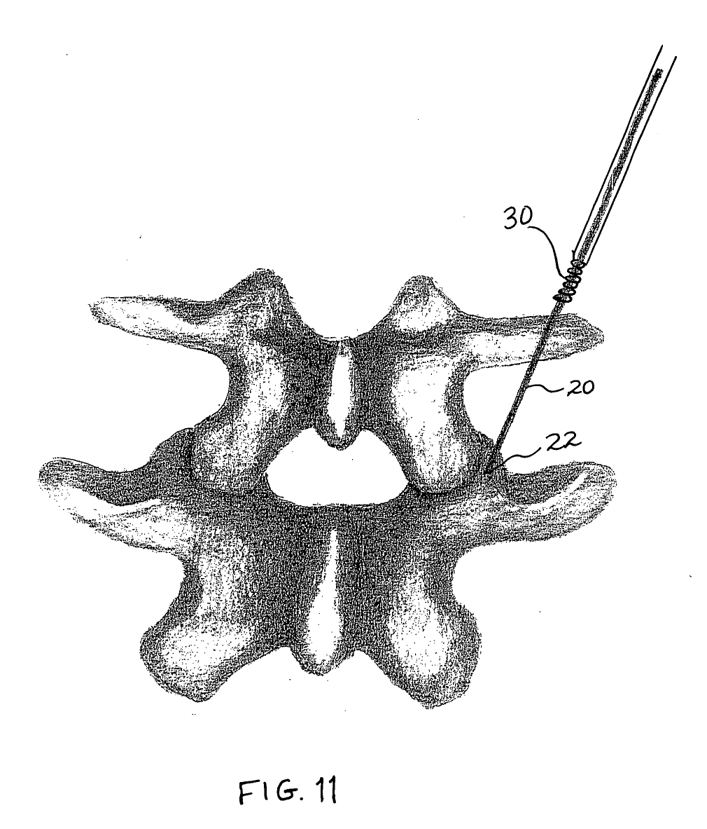

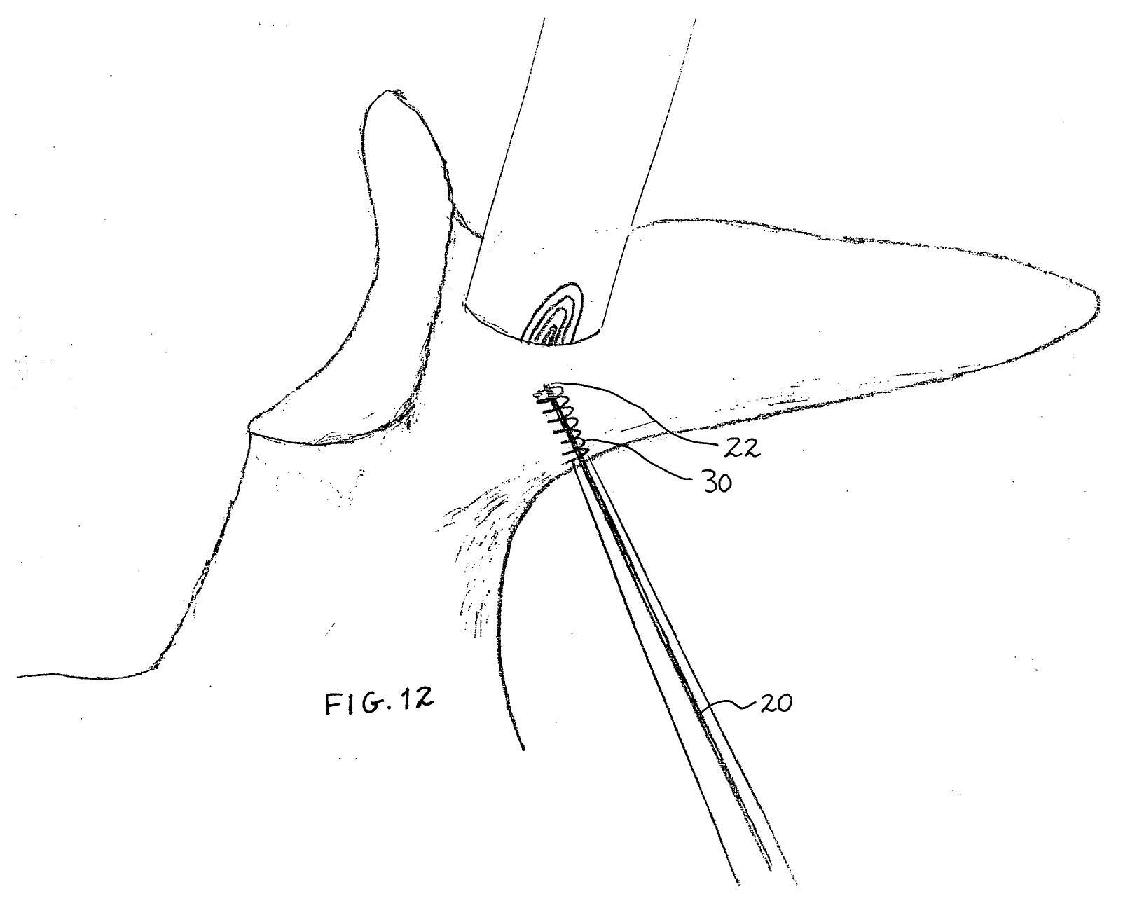

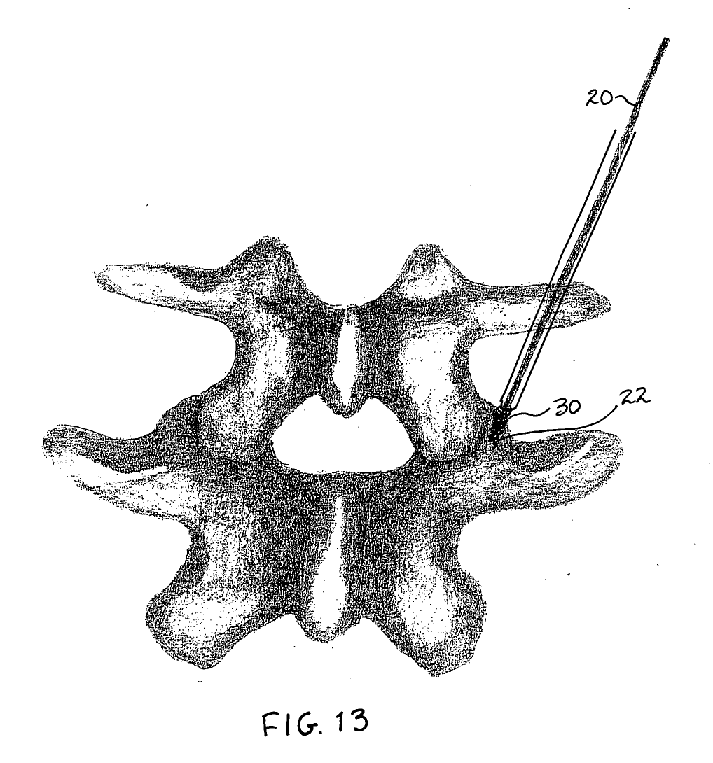

With the guide needle 20 in place, a cannulated hand drill 30 is passed over the guide needle 20, as seen in FIGS. 10 and 11. The drill 30 is advanced to the fusion site 22, and then operated to enlarge the hole in the bone of the transverse process 18, as seen in FIGS. 12 and 13. Of course, while the drill is utilized to enlarge the hole, it is not a necessary component of the invention, and merely facilitates the use of handsaw 32. If the surgeon decides to use another cutting means, such as laser, electrocautery, etc., the drill may not be needed.

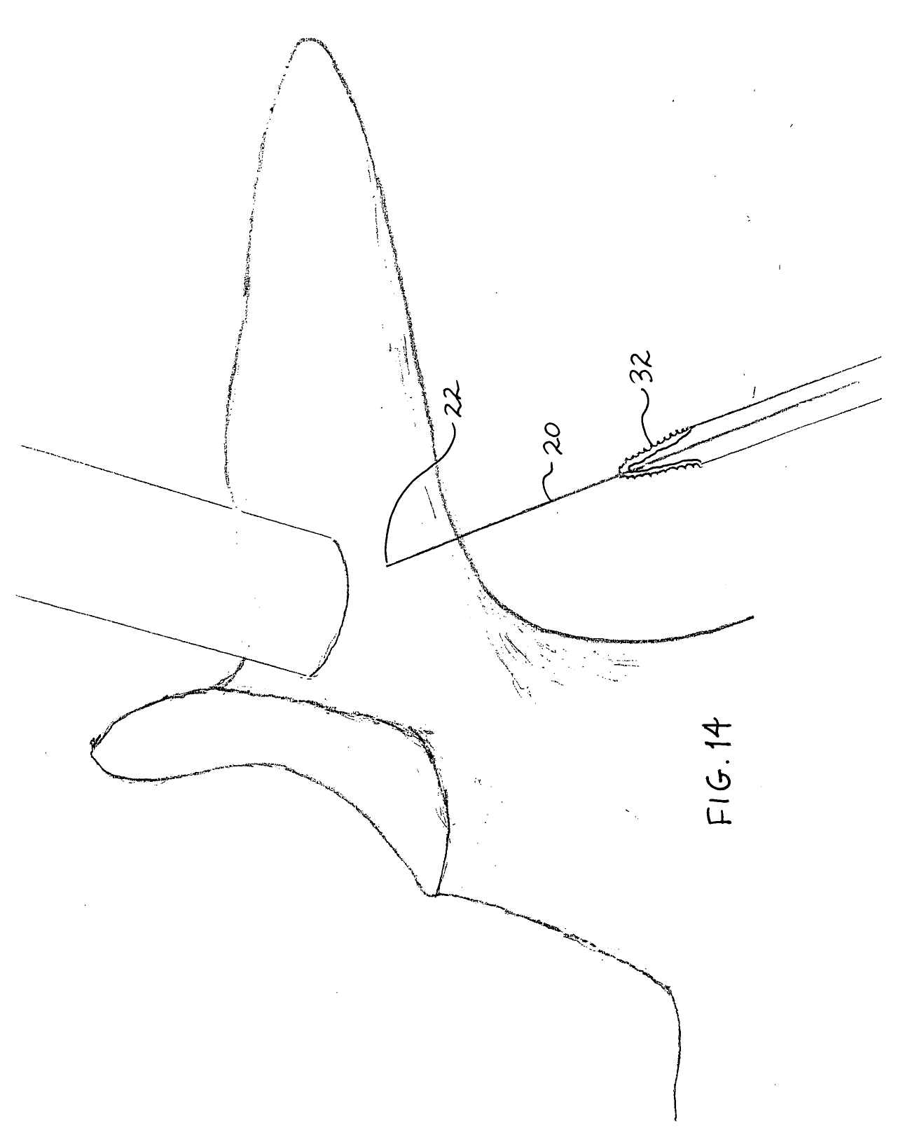

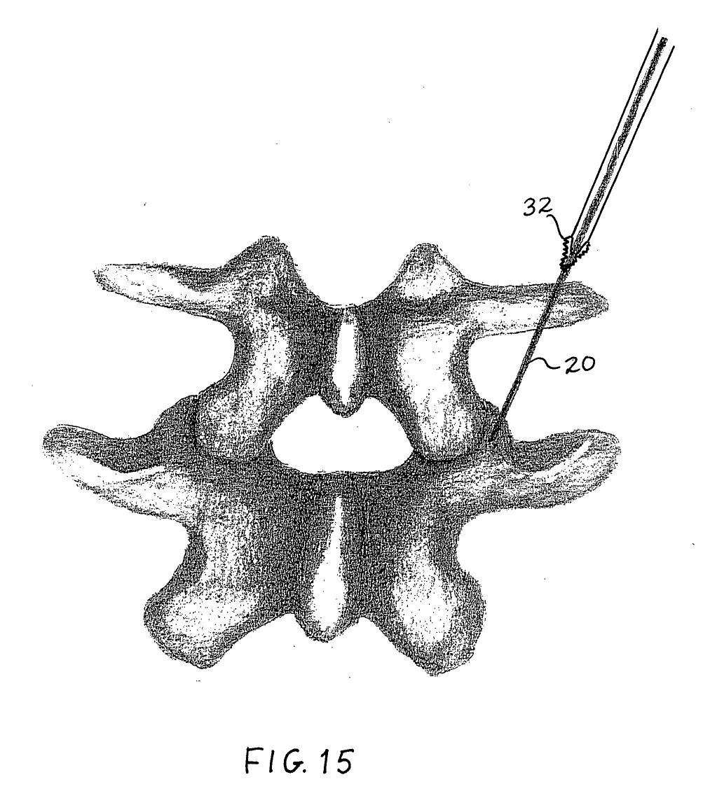

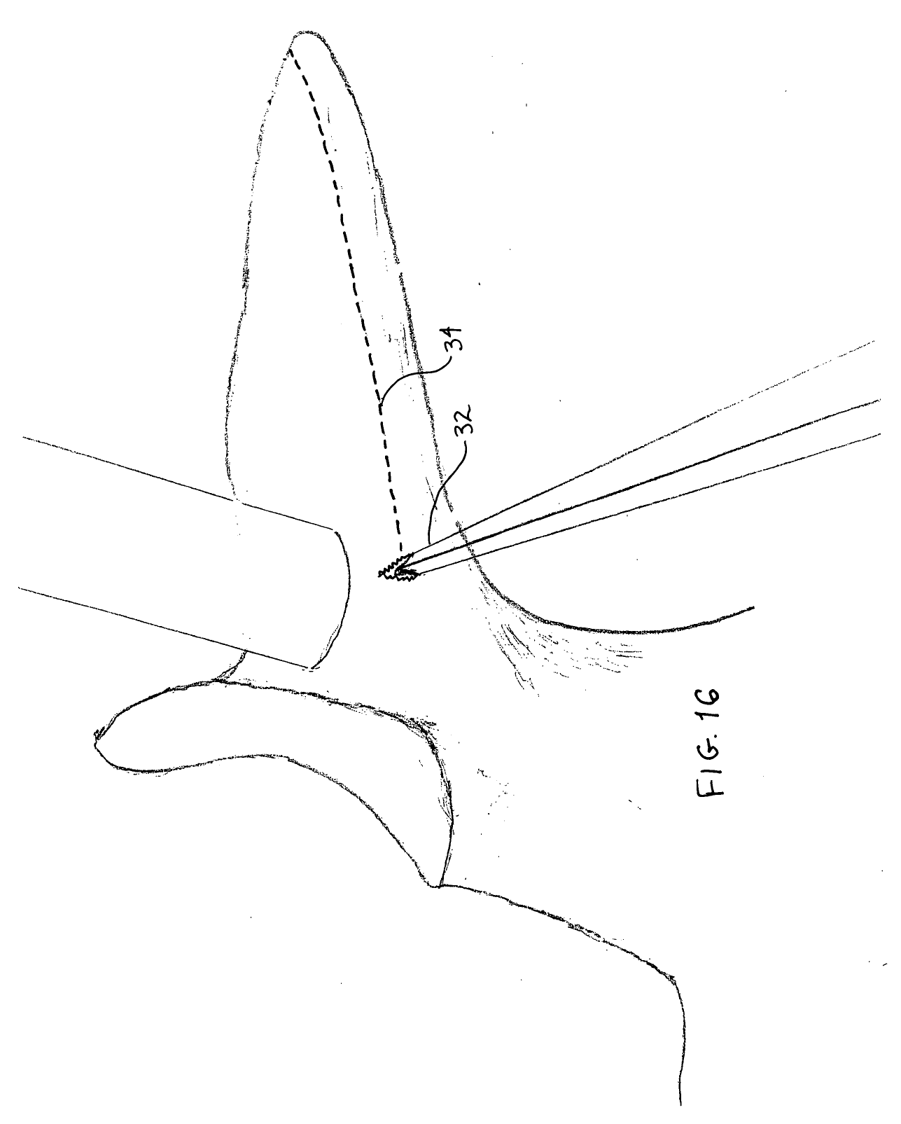

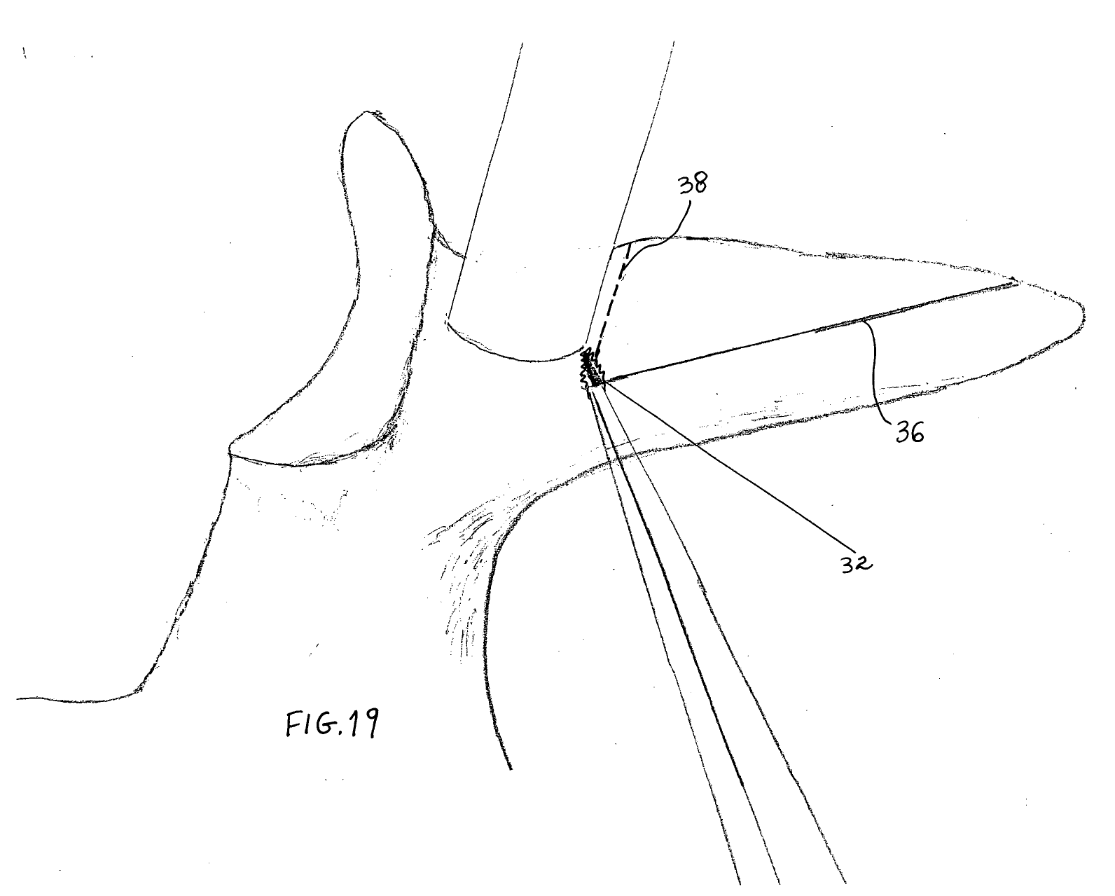

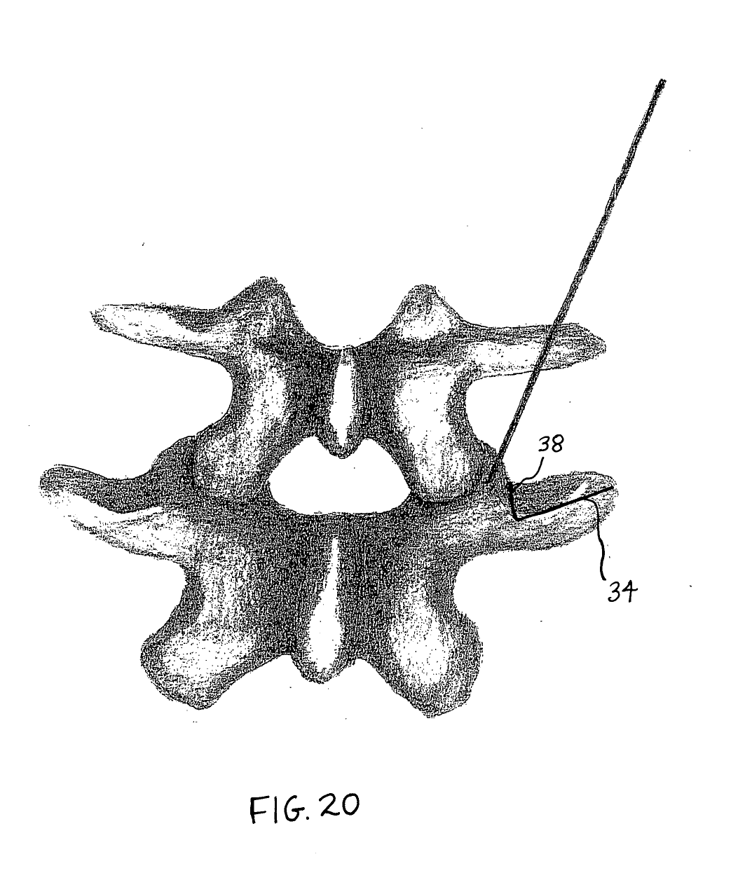

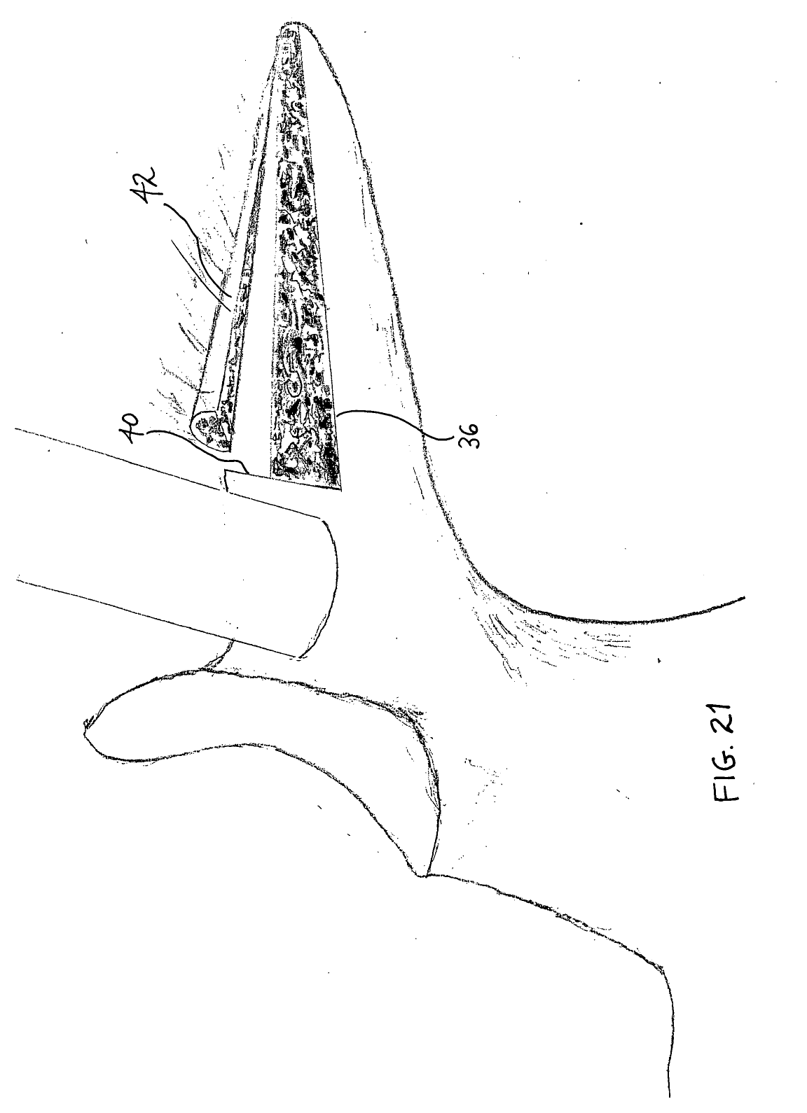

Once the drilling operation is complete, the drill 30 is removed, and cannulated hand saw 32 is passed over the guide needle 20 and advanced to the hole made by the drill 30, as seen in FIGS. 14 and 15. Once the tip of the saw 32 reaches the hole at the fusion site 22, the saw is operated to cut the transverse process 18 in the coronal plane along the proposed course 34 to make the cut 36 through the bone of the transverse process 18, as seen in FIGS. 16-18. After cut 36 is complete, the saw 32 is repositioned at the origin of cut 36 and rotated 90° to make a vertical cut along proposed course 38, as seen in FIGS. 19 and 20. After cut 40 is made, and the osteotomy is completed as seen in FIG. 21, bone flap 42 is separated from transverse process 18, and the saw 32 is removed. Bone flap 42 remains attached to the muscles, and remains vascularized to provide a bed for the fusion.

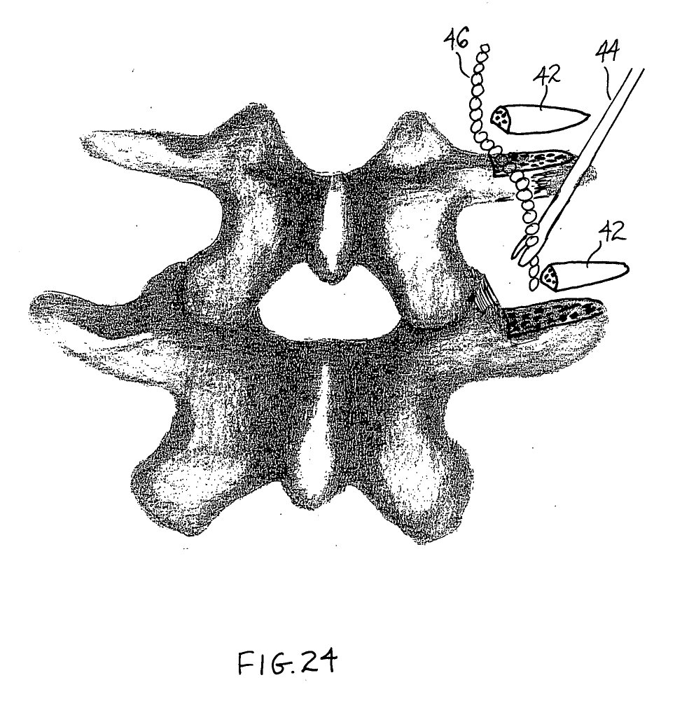

As described above, the cutting procedure is performed on the inferior vertebrae 14. Following this, the procedure is repeated on the superior vertebrae 12, although the order may of course be reversed. The completed osteotomies, performed on both the inferior vertebrae 14 and the superior vertebrae 12, are shown in FIG. 24.

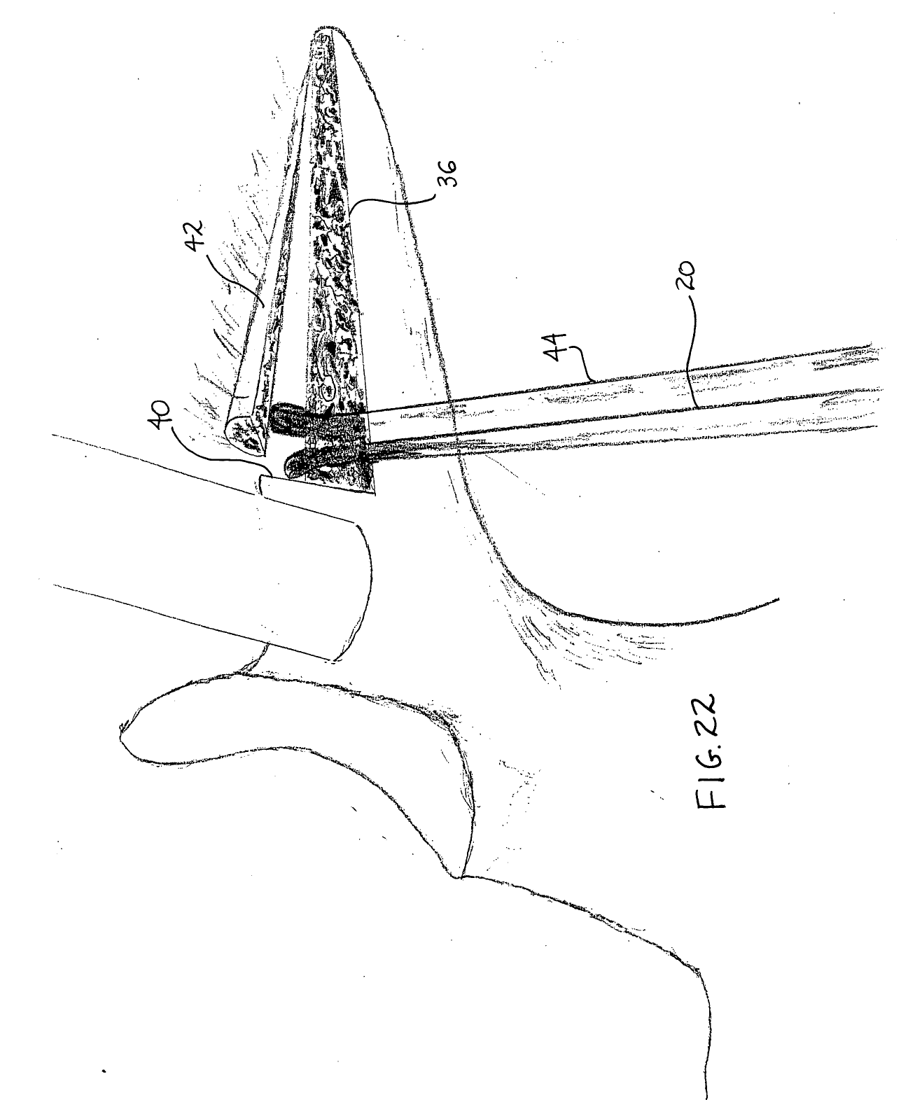

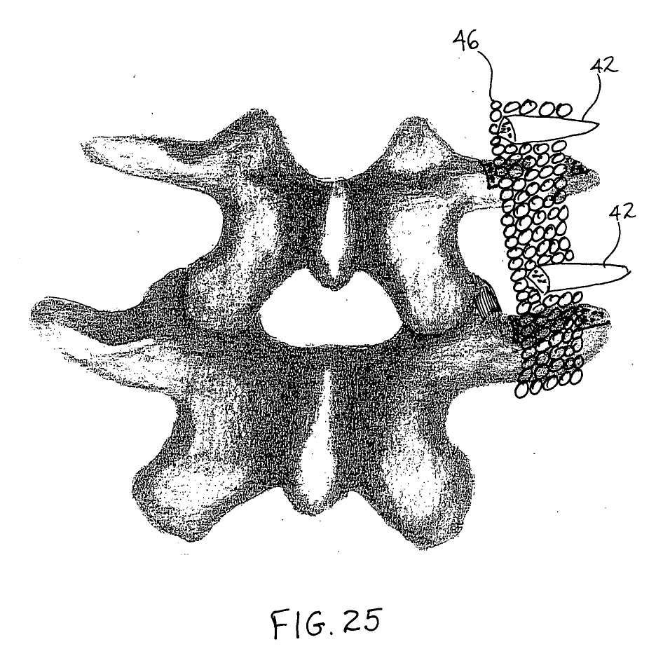

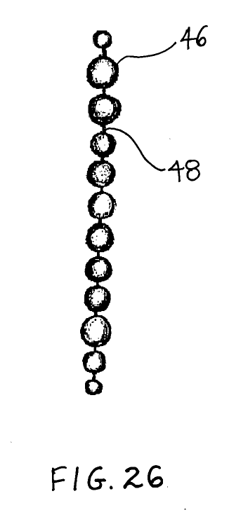

After the saw is removed, an instrument, such as grasping tool 44, is inserted to lift the bone flap 42 off the transverse process 18. The bone flap 42 is still connected to its corresponding muscles, and is merely pushed out of the way, as seen in FIG. 22. At this point, bone graft material, such as the bone graft capsules 46 of the present invention, are introduced to the site and are placed over the transverse processes 18, between the transverse processes 18 and the bone flaps 42. The grasping tool may be utilized to manipulate the capsules 46 into place, as seen in FIG. 25, and multiple strings of the capsules 46 moved into place between the transverse processes 18 and the bone flaps 42, as seen in FIG. 26, to create a bony bridge between the transverse processes 18. Due to the confined space in which the transverse processes and bone flaps are located, the bone flaps merely rest on the capsules 46 as the muscles to which the bone flaps 42 are attached return to their original position with respect to their respective transverse processes 18. If desired, the bone flaps could be further secured to the transverse processes 18, for example, by bone glue.

Once the bone graft capsules 46 are in place, the instruments are removed, and the incisions are closed.

The bone graft capsules 46, as seen in FIGS. 27 and 28, are preferably in the form of a string of capsules, connected by a bioabsorbable string 48. The capsules contain bone fragments 50 and are enveloped by a gelatinous material, which is readily absorbed into the body once implanted. The bone fragments 50 may be cadaveric bone, morselized bone harvested from the patient prior to the fusion procedure, demineralized bone, and partially demineralized bone, preferably suspended in a bioabsorbable matrix. Bone growth material is also preferably contained in the capsules. Bone growth materials that may be used in conjunction with the capsules of the present invention include bone morphogenic protein (BMP) material, bone gel, methylmethacrylate, hydroxyapatite, or any other biocompatible, bioabsorbable substance. Although shown as spheres, the capsules can be of any suitable shape.

While the invention has been shown and described with reference to certain preferred embodiments, it will be understood by those skilled in the art that various changes and modifications in form and detail may be made therein without departing from the spirit and scope of the invention, as defined by the appended claims.

1. A bone graft delivery system, comprising:
a plurality of capsules joined together, each capsule containing bone fragments; and
a coating on each of the capsules to enclose the bone fragments, the coating being made of bioabsorbable material.

2. A method for performing a fusion procedure between the transverse processes of two adjacent vertebra, comprising:
making an incision;
inserting a guide needle through the incision to the fusion site;
inserting cutting tool over the guide needle to the transverse process of a first vertebrae;
cutting a first portion of the transverse process of the first vertebrae in a coronal plane;
cutting a second portion of the transverse process at a 90° angle to the first portion to create a vascularized bone flap;
relocating the guide needle and cutting tool to the transverse process of the second vertebrae;
cutting a first portion of the transverse process of the second vertebrae in a coronal plane;
cutting a second portion of the transverse process at a 90° angle to the first portion to create a vascularized bone flap;
removing the cutting tool and guide needle;
introducing a bone graft delivery device to the two transverse processes between the vascularized bone flaps and the transverse processes; and
closing the incision.
